(19) United States
(12) Patent Application Publication
(10) Pub. No.: US 2009/0275114 A1
(43) Pub. Date: Nov. 5, 2009

(54) VIBRATORY MIXER

(76) Inventor: Marcel Roll, Maur (CH)

Correspondence Address: George Pappas;Harrison Place, 919 S Harrison Street, Suite 300, Fort Wayne, IN, 46802, US

(21) Appl. No.: 12/312,499

(22) PCT Filed: Oct. 23, 2007
(86) PCT No.: PCT/CH2007/000528

§ 371 Date: Jun. 22, 2009

(30) Foreign Application Priority Data
Nov. 14, 2006 (CH) 1827/06

(51) Int. Cl. C12M 1/36; C12M 1/10; B01J 19/28; C12M 1/34; C12M 1/12; G01N 35/00

(52) U.S. Cl. 435/286.7; 435/298.2; 422/209; 435/287.1; 422/68.1; 435/297.2; 422/105

(57) ABSTRACT

To simplify mixing systems without stirrer systems and to minimize cleaning and validation operations, it is proposed that a vibratory mixer with a vibratory mechanism should have a container. Into this container is inserted a bag which, after use, need not be cleaned and is instead replaced by a new one. This bag is equipped with the connections and sensors required and presterilized. The bag is placed into an angled container. The container serves as a stabilization casing. As a result of the vibration of the container, there is liquid displacement over the angle range, which improves the mixing operation.

This application claims priority of PCT application PCT/CH2007/000528 having a priority date of Nov. 14, 2006, the disclosure of which is incorporated herein by reference.

### TECHNICAL FIELD

The invention relates to a rocking mixer. The rocking mixer is particularly suitable as what is known as a disposable mixer for biological, chemical and fermentation mixing processes for a volume of 10 to 1000 liters.

### BACKGROUND OF THE INVENTION

Rocking mixers of the generic type mentioned above have been known for a long time, for example from WO-A-2000/066706 or else, in a simpler way, in U.S. Pat. No. 6,190,913 B1 or in US-A-2003/036192. In all these publications, it is proposed for a bioreactor or rocking mixer to arrange a plastic bag on a container or on a rockable plate, to fill it with the treatment stock and to intermix the capacitys of the bag by means of a rocking action of the plate and consequently cause them to react.

### SUMMARY OF THE INVENTION

The object of the invention is to further improve a Rocking mixer of the type mentioned.

Since the container has an angled region which coincides with the rocking axis or is essentially parallel to this, the bearing surface is subdivided into two subsurfaces which, moreover, form an angle of 60° to 120°. This leads to an improved intermixing which can also be controlled more effectively.

Any deviations of the angled region from the rocking axis may be 15° to 20°, preferably below 5°.

The rocking angle and the rocking speed can be adapted to the optimal process conditions (mixing) by the means for setting the rocking drive. It became clear that rocking of 50°-60° on both sides proves to be advantageous for the mixing process. The entire drive should therefore be rockable by at least up to 120°. The rocking axis may advantageously intersect the center of gravity resultant of the container, this being shown to be advantageous for the drive power.

According to the invention, the container is angled at 60° to 120°. It was shown, however, that an angle of 90° proves to be ideal for the mixing process. The manufacturing material for the container may advantageously be chrome steel. However, a plastic may also be used. The container should have a volume of at least 50 liters. At the leg ends of the container, a protuberance is provided which serves as a liquid reservoir, but at the same time also prevents the displacement of the bag in the container when the latter is moved over the horizontal position. The angled region of the container subdivides the bag into two compartments. The angled region may have a horizontal surface on which flow breakers in any desired form are attached or incorporated. These flow breakers result in an additional swirling of the liquid during overflow.

By a vertically adjustable transverse strut being mounted directly above the angled region, the overflow velocity of the liquid at the flow breakers can be set.

To fasten the bag, advantageously, fastening bolts in which the bag is suspended may be attached to at least one end, expediently to both ends of the angled region.

The bag advantageously consists of a plastic film preferably composed of PE, EVA or polyester. The form of the bag is in this case adapted to the internal form or the volume of the container. The bag length extends over the entire angled length of the container.

It is advantageous if the bag is equipped with a disposable pH probe and, if required, also further disposable probes for monitoring the process.

Tabs are attached on both sides along the center line of the bag and have position holes which serve for the exact positioning and fastening of the bag in the container. It is advantageous to ensure an additional introduction of gas into the liquid by detachment of a filter fabric in the internal region of the bag, said filter fabric being positioned on the two leg sides, and by the feed of a gas between the filter fabric and the vertical-overflow filter surface (waterfall effect).

The rocking mixer is particularly advantageous if it additionally has a device for determining the capacity of the bag, in order thereby to control the rocking angle and/or the rocking speed. To be precise, if the bag has a large capacity, then only a relatively low rocking angle is necessary so that a mixing operation can be carried out. If, however, for example, a large part of the capacity has already been extracted, then the remainder remains in the lower region, and a high rocking angle is necessary in order to bring this remainder over the bend region. The device may simply be a management system, by means of which the loading of the bag and extraction are processed. Advantageously, however, also at least one weight sensor for measuring the weight of the container (gross) and consequently of the bag capacitys (net) is used, which is advantageously arranged in the vicinity of or below the bend region.

The elements to be used according to the invention, which are mentioned above, and also those claimed and described in the following exemplary embodiments are not subject to any special exceptional conditions in terms of their size, shape, use of material and technical design, and therefore the selection criteria known in the respective field of use may be employed unrestrictedly.

### BRIEF DESCRIPTION OF THE DRAWINGS

Exemplary embodiments of the invention are described in more detail below with reference to the drawings in which:

FIG. 1 shows a rocking mixer in the neutral position, illustrated from the side;

FIG. 2 shows a container for the rocking mixer of FIG. 1;

FIG. 3 shows the rocking mixer of FIG. 1 in a position pivoted to the left, the right container part being in a horizontal position;

FIG. 4 shows the rocking mixer of FIG. 1 in a position pivoted to the right, the left container part being in a horizontal position;

FIG. 5 shows a bag (mixing bag) for the container of FIG. 1, in a view from above;

FIG. 6 shows the bag (mixing bag) for the container from FIG. 1, in a view from the side;

FIG. 7 shows the operating principle of the flow breaker for the rocking mixer of FIG. 1; and

FIG. 8 shows the rocking mixer of FIG. 1 in a perspective illustration.

### DETAILED DESCRIPTION OF THE PREFERRED EMBODIMENTS

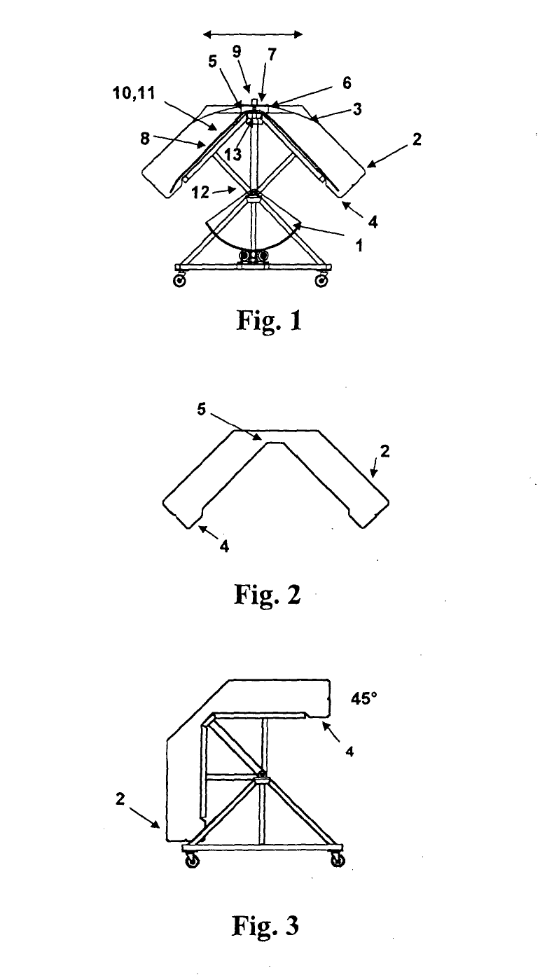

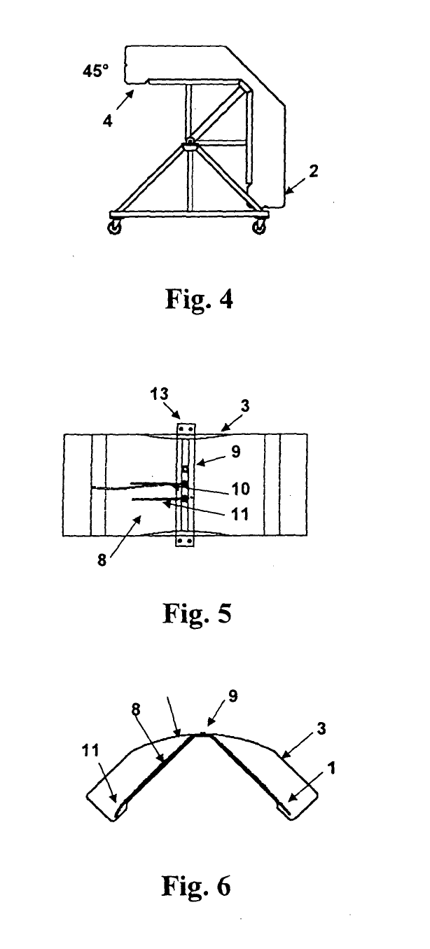

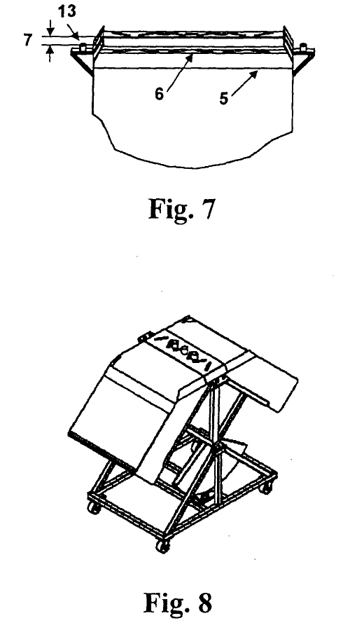

The rocking mixer according to FIGS. 1 to 8 is a 50 to 1000 liter media and cultivation mixing system and is based on induced liquid rearrangement. The system has a rocking mechanism 1 which can execute a rocking movement extending up to 120 degrees. Mounted on this rocking mechanism is an angled container 2, at which a bag 3 adapted to the container form, in the present exemplary embodiment a plastic bag, is introduced. The container is preferably angled from 60° to 120°, and 90° in the exemplary embodiment illustrated in the figures; this affords the shape important to the mixing process. The bag 3 preferably consists of a plastic film composed of polyethylene, EVA or polyester or of a similar material and is introduced into the container 2. The bag 3 serves additionally as a disposable sheath which in the present exemplary embodiment is presterilized. The entire mixing and reaction process takes place in this bag 3 and eliminates the need for cleaning the container 2 after the end of the process.

The bag is equipped with a screw fastening 9 and a connecting hose 10, furthermore with an extraction hose 11 having an internal suction hose and with a sampling connection. A disposal probe for measuring the pH value and further disposable probes serve for monitoring the process.

The container 2 is provided with protuberances 4 at the ends, so that the bag 3, with the liquid collecting in these protuberances, is fixed and at the same time forms a liquid reservoir, out of which liquid can be extracted continuously during rocking.

Owing to the rocking of the liquid-filled bag in the container 2 up to 60° out of the neutral position on both sides, so that one container part or the other is alternately brought into the horizontal position, with a rocking of 45° in each case in the exemplary embodiment described here, a liquid overflow occurs in the angled region 5. The liquid can therefore be mixed in a controlled manner by means of the adjustable rocking speed.

The setting of the rocking speed and the attachment of flow breakers 6 in the angled region 5 and also the narrowing of the bag cross section 7 above the angled region give rise to a greater or lesser swirling (mixing). The acceleration of the liquid over the angled region 5 and renewed braking on the bottom of the container 2 produce further mixing turbulences important for the process.

The rearrangement of the entire liquid per rocking unit generates a maximum mixing value, in conjunction with a disposable bag, and therefore maximum sterility and cleaning performance are also ensured.

Attaching a filter fabric 8 in the internal region of the bag, which filter fabric is positioned on both leg sides, makes it possible to feed a gas between the filter fabric and the vertical-overflow filter surface. An additional introduction of gas into the liquid can consequently be ensured (waterfall effect).

In addition, the rocking mixer has a device for determining the capacity of the bag, in order thereby to control the rocking angle and/or the rocking speed. If the bag has a large capacity, a relatively low rocking angle is predetermined in order to carry out a mixing operation. If, however, the bag has only a small capacity, a high rocking angle is predetermined in order to bring this remainder of the mixing stock over the angled region. In the present exemplary embodiment, the rocking mixer has a weight sensor which is arranged in the vicinity of or below the angled region.

In the present exemplary embodiment, the subsurfaces of the container can be heated individually so that the reaction parameters can be controlled or regulated.

### LIST OF REFERENCE SYMBOLS

1 Rocking mechanism

2 Container

3 Bag

4 Protuberance

5 Angled region

6 Flow breaker

7 Bag cross section

8 Filter fabric

9 Screw connection

10 Connecting hose

11 Extraction hose

12 Center of gravity resultant

13 Fastening bolt

1. A rocking mixer for biological, chemical and fermentation mixing processes, with a reactor stand and with a container, the container having a bearing surface on which a bag lies, the container being rockable about an essentially horizontal rocking axis of the reactor stand by means of a rocking mechanism, and the rocking mixer having means for setting the magnitude of the rocking angle, characterized in that the container has at least one angled region which coincides with the rocking axis or is essentially parallel to this and which subdivides the bearing surface into two subsurfaces which are at an angle of 60° to 120° with respect to one another.

2. The rocking mixer as claimed in claim 1, characterized in that the container is rockable by the rocking mechanism at an angle of at least 90°, preferably of 120°.

3. The rocking mixer as claimed in claim 1, characterized in that the rocking axis intersects the center of gravity resultant of the container.

4. The rocking mixer as claimed in claim 1, characterized in that the subsurfaces due to the angled region have an angle of approximately 90°.

5. The rocking mixer as claimed in claim 1, characterized in that a protuberance which forms a liquid reservoir is provided at the leg ends of the container.

6. The rocking mixer as claimed in claim 1, characterized in that the angled region of the container is designed such that it subdivides the bag into two compartments.

7. The rocking mixer as claimed in claim 1, characterized in that the angled region has a horizontal surface.

8. The rocking mixer as claimed in claim 1, characterized in that flow breakers are attached or incorporated in the region of the angled region.

9. The rocking mixer as claimed in claim 8, characterized in that a vertically adjustable transverse strut is arranged in the region of the angled region.

10. The rocking mixer as claimed in claim 1, characterized in that fastening bolts in which the bag can be suspended are arranged at at least one end of the angled region.

11. The rocking mixer as claimed in claim 1, characterized in that the bag has at least one disposable pH probe for monitoring the mixing process.

12. The rocking mixer as claimed in claim 1, characterized by tabs with position holes, which are formed on both sides on the center line of the bag, for positioning and fastening the bag in the container.

13. The rocking mixer as claimed in claim 1, characterized by a filter fabric in the internal region of the bag, which filter fabric is preferably positioned on the two leg sides, and by a device for feeding a gas between the filter fabric and the vertical-overflow filter surface.

14. The rocking mixer as claimed in claim 1, characterized by a device for determining the capacity of the bag, by means of which device the rocking angle and/or the rocking speed can be controlled.

15. The rocking mixer as claimed in claim 14, characterized in that the device comprises a management system, by means of which the loading of the bag and the extraction of the bag can be processed.

16. The rocking mixer as claimed in claim 14, characterized in that the device comprises a weight sensor for measuring the weight of the container and/or of the bag.

17. The rocking mixer as claimed in claim 2, characterized in that the rocking axis intersects the center of gravity resultant of the container.

18. The rocking mixer as claimed in claim 3, characterized in that the subsurfaces due to the angled region have an angle of approximately 90°.

19. The rocking mixer as claimed in claim 11, characterized in that the device comprises a weight sensor for measuring the weight of the container and/or of the bag.

20. The rocking mixer as claimed in claim 15, characterized in that the device comprises a weight sensor for measuring the weight of the container and/or of the bag.
